(19) United States
(12) Patent Application Publication
(10) Pub. No.: US 2006/0149184 A1
(43) Pub. Date: Jul. 6, 2006

(54) Myocardial stimulation

(76) Inventors: Orhan Soykan, Shoreview, MN (US); D. Curtis Deno, Andover, MN (US)

Correspondence Address: MEDTRONIC, INC., 710 MEDTRONIC PARK, MINNEAPOLIS, MN, 55432-9924, US

(21) Appl. No.: 11/030,574

(22) Filed: Jan. 6, 2005

(51) Int. Cl. A61N 1/30

(52) U.S. Cl. 604/20

(57) ABSTRACT

In general, the invention is directed to methods and devices for electrically stimulating heart tissue. The invention includes delivery of stimulation to transplanted biological material, such as transplanted cells, transplanted in a myocardium of a heart during an ejection phase of a cardiac cycle. The invention also includes delivery of cardiac potentiation therapy stimulation, which improves the hemodynamic performance of the heart. Stimulation to transplanted biological material and cardiac potentiation therapy stimulation can improve the performance of a heart damaged by myocardial infarction.

### TECHNICAL FIELD

The present invention relates to systems and methods and implantable systems associated with the heart, and in particular, to systems and methods associated with stimulating the myocardium.

### BACKGROUND

During coronary artery disease, formation of plaques narrows the lumen of the coronary artery, reducing the O₂ supply to cardiac tissue. If the coronary artery becomes occluded, the cardiac tissue served by the coronary artery soon dies from O₂ deprivation. Actual necrosis of heart tissue is called acute myocardial infarction, or heart attack.

Once the cardiac tissue has died, the tissue becomes infiltrated with noncontracting scavenger cells, which are ultimately replaced with fibrous scar tissue. The fibrous scar tissue, which includes fibroblasts and an extracellular matrix, does not significantly contribute to the contraction of the heart. Cardiac cells do not naturally repopulate the damaged region.

A consequence of myocardial infarction is a loss of hemodynamic function. With a loss of tissue contributing to the pumping of blood, the patient may experience reduced cardiac output and reduced systolic blood pressure. Numerous morbid conditions are sequelae of the loss of hemodynamic function.

Cellular cardiomyoplasty involves transplanting cells into the damaged myocardium to repopulate the damaged region. In one procedure, cells are transplanted by injection directly into or proximate to the affected tissue. The transplanted cells are more elastic than the fibrous scar tissue, and therefore the presence of the cells enhances the elasticity of the heart. The elasticity provided by the cells improves the performance of the heart during diastole, which is the relaxing and filling phase of the cardiac cycle.

### SUMMARY

In general, the invention is directed to methods and devices for electrically stimulating tissue to improve hemodynamic function. Stimulation of transplanted biological material, such as transplanted cells, can improve the performance of the heart during systole. In addition, the invention is directed to methods and devices for applying cardiac potentiation therapy (CPT), i.e., electrically stimulating one or more heart chambers to induce post-extrasystolic potentiation. These therapies can improve hemodynamic function for a patient that has lost function due to myocardial infarction.

Stimulation of biological material transplanted in a myocardium of a heart during an ejection phase of a cardiac cycle can improve hemodynamic function. The transplanted biological material may include cells, such as skeletal myoblasts, precursor cells, endothelial cells, differentiated or undifferentiated stem cells, undifferentiated contractile cells, fibroblasts and genetically engineered cells. The biological material may further comprise components of cells, such as genetic material, or a chemoattractant to attract precursor cells. Some of the biological material, such as skeletal cells, may be naturally contractile. It has been discovered that electrical stimulation may result in differentiation or phenotypic conversion, causing the biological material to become more contractile.

In a typical application, an implantable medical device (IMD) delivers a set of stimulating pulses to the transplanted biological tissue when contraction of the transplanted tissue will assist in hemodynamic function. In general, stimulation during the ejection phase of the cardiac cycle, when the aortic and pulmonary valves are open, provides hemodynamic assistance.

The invention encompasses various techniques for stimulating the biological material during the ejection phase. The IMD may, for example, time the delivery of the stimulations by observing an electrical signal generated by the heart, such as an R-wave. In some embodiments, the IMD may deliver pacing stimulations to the heart, and the IMD may time the delivery of the stimulations according to the paces. The IMD may also time the delivery of the stimulations according a biological signal detected by a sensor, such as a sound sensor, pressure sensor, impedance sensor, flow meter or accelerometer.

CPT improves hemodynamic function by inducing post-extrasystolic potentiation. In particular, CPT involves managing the distribution of calcium ions that contribute to contraction of cardiac myocytes by stimulating active pumps that take up calcium ions from the cytosol into the sarcoplasmic reticulum. CPT stimulations are extrasystolic, in that they are delivered prematurely, i.e., in advance of a stimulation that would result from a normal cardiac rhythm. CPT stimulations are typically delivered at a time when the heart is not ready to contract, so CPT stimulations do not cause contraction to result. CPT stimulations do, however, increase the take up of calcium ions by the sarcoplasmic reticulum. Because of this management of the distribution of calcium ions, the cardiac myocytes relax more fully during diastole, thereby improving diastolic filling, and the cardiac myocytes contract more forcefully during systole, thereby increasing cardiac output and systolic pressure.

A patient that has suffered a myocardial infarction can benefit from stimulations to transplanted biological material in concert with CPT stimulations. A patient that has suffered a myocardial infarction may have lost hemodynamic function, and the stimuli can compensate for that loss of function. Stimulations applied to the transplanted biological material may also influence the transplanted biological material to contribute to pumping, and CPT stimulations enhance cardiac filling and improve the forcefulness of the cardiac contractions. These stimulations contribute to improvement of hemodynamic function.

In one embodiment, the invention is directed to a method comprising electrically stimulating biological material transplanted in a myocardium of a heart during an ejection phase of a cardiac cycle. The transplanted biological material may be in, or proximate to, an infarct region of the myocardium. The stimulation may include a set of one or more stimulating pulses.

In another embodiment, the invention is directed to a method that comprises electrically stimulating biological material transplanted in a myocardium of a heart during an ejection phase of a cardiac cycle, and electrically stimulating a chamber of the heart to induce post-extrasystolic potentiation. This method applies the stimulation therapies with the goal of improving the hemodynamic performance of the heart. In another embodiment, the invention is directed to a computer-readable medium comprising instructions for causing a programmable processor to carry out the methods of the invention.

In a further embodiment, the invention is directed to a system that includes a first electrode configured to deliver a first electrical stimulation to biological material transplanted in a myocardium of the heart and a second electrode configured to deliver a second electrical stimulation to a chamber of a heart. The system also includes a processor configured to control delivery of the first stimulation during an ejection phase of a cardiac cycle and further configured to control delivery of the second stimulation to induce a post-extrasystolic potentiation. The system may also include one or more sensors to sense one or more biological signals, and may also include the capability of delivering defibrillation therapy.

The invention may result in one or more advantages. Development of necrotic tissue causes the heart to become less elastic, and also can adversely affect hemodynamic function. It has been observed that biological material transplanted in or proximate to an infarct region of a heart improves the elasticity of the heart. Also, stimulation of the biological material according to the invention can improve the hemodynamic function of the heart, by causing contraction of at least a portion of the biological material, thereby contributing to the ejection of blood. Furthermore, application of the stimulation to the biological material may speed up the formation of the contractile tissue and prevent the invasion of the infarct region by non-contractile fibroblasts. CPT stimulations provide benefits as well, by improving cardiac filling during diastole and the forcefulness of contraction during systole. CPT stimulations can help compensate for a loss of hemodynamic function that follows from a myocardial infarction.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is an illustration of a human heart showing deployment of leads and electrodes according to an embodiment of the invention.

FIG. 2 is a block diagram showing a system that can electrically stimulate biological material transplanted in a myocardium and that can apply cardiac potentiation therapy.

FIG. 3 is a timing diagram illustrating signals and the timing of electrical stimulations according to various embodiments of the invention.

FIG. 4 is another timing diagram illustrating signals and the timing of electrical stimulations according to various embodiments of the invention.

FIG. 5 is timing diagram illustrating a set of pacing stimulations and a corresponding response of transplanted biological material having contractile properties of skeletal muscle.

FIG. 6 is a flow diagram illustrating a technique for timing the delivery of electrical stimulations to transplanted biological material and the delivery of cardiac potentiation therapy.

### DETAILED DESCRIPTION

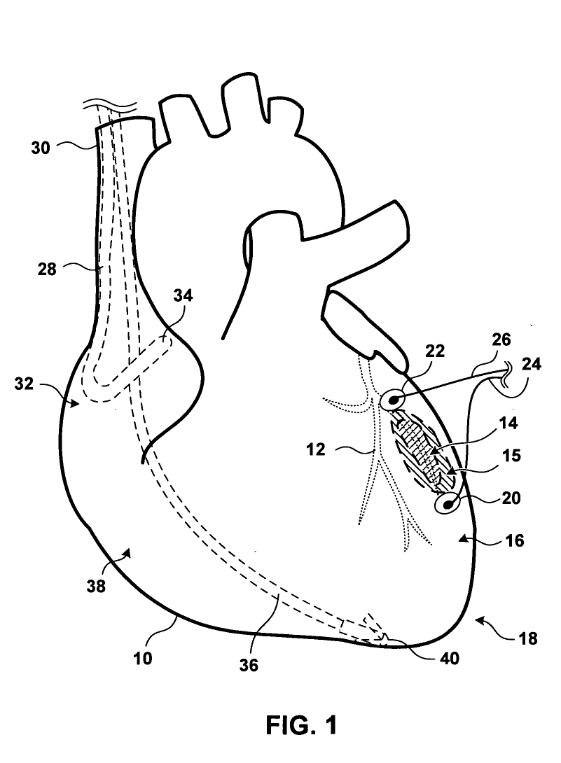

FIG. 1 is a schematic diagram of a human heart 10. A blockage in a branch of coronary artery 12 has deprived region 14 of a blood supply, and consequently of oxygen. As a result, the myocardial tissue in region 14 has become damaged. In particular, some tissue has become necrotic, and an infarct region 14 has developed. In the example shown in FIG. 1, infarct region 14 is on the epicardium of the left ventricle 16.

Necrotic tissue does not contribute to the pumping action of heart 10. In particular, infarcted tissue does not contract in response to the excitation that takes place during a cardiac cycle. Normally, a ventricular excitation propagates from proximate to the apex 18 throughout the ventricular myocardium via gap junctions in the cardiac muscle, and the cardiac muscle contracts. The excitation does not cause infarct region 14 to contract, however. On the contrary, infarct region 14 can disrupt the propagation of the excitation, thereby affecting the excitation of healthy cardiac muscle. Moreover, scar tissue in infarct region 14 is usually less elastic than cardiac muscle, and can impair the function of heart 10 during the systolic and diastolic phases.

In the example of FIG. 1, a zone 15 proximate to infarct region 14 has been repopulated with transplanted biological material. The biological material, which may be transplanted into, transplanted proximate to or transplanted around the necrotic tissue, may include any of several biological substances, singly or in combination. The biological material may include cells, such as skeletal myoblasts, precursor cells, endothelial cells, differentiated or undifferentiated stem cells, undifferentiated contractile cells, fibroblasts and genetically engineered cells. The biological material may further comprise components of cells, such as genetic material, genetic vectors such as viruses, or proteins such as Insulin-Like Growth Factor or other growth factors. The biological material may also include a chemoattractant to attract precursor cells from the heart or from the other organs to repopulated zone 15 or infarct region 14. These categories of biological material are not exclusive of one another, and a particular element of biological material may belong to more than one category. Also, the transplanted biological material need not be exclusively biological, but may include an inorganic or engineered material, such as a scaffold to hold biological material. Furthermore, the invention is not limited to the particular materials listed herein.

Nor is the invention limited to any particular transplantation technique. For a typical patient, a surgeon may transplant biological material by injection during a surgical procedure, such as an open-heart procedure. The surgeon may inject the biological material into the necrotic tissue or proximate to the necrotic tissue. The surgeon may also deliver the biological material through the coronary vasculature. In practice, implanted cells have been observed to migrate, so over time some biological material transplanted in infarct region 14 may migrate outside infarct region 14. In addition, biological material transplanted in infarct region 14 may migrate to a different site inside infarct region 14.

In FIG. 1, repopulated zone 15 surrounds infarct region 14, and can include part or all of infarct region 14. In a typical patient, repopulated zone 15 may have a perimeter about a centimeter (0.4 inch) around the region of necrotic tissue. The invention is not limited to cases in which repopulated zone 15 completely surrounds infarct region 14, however.

At least two electrodes 20 and 22 are deployed epicardially proximate to repopulated zone 15. In particular, electrodes 20 and 22 are deployed such that a line between electrodes 20 and 22 substantially follows direction of contraction of natural cardiac muscle fibers. Electrodes 20 and 22 are deployed so that an electrical stimulation delivered to the epicardium via electrodes 20 and 22 creates a difference in electrical potential, which in turn generates an electrical field that captures contractile fibers in repopulated zone 15. In other words, electrodes 20 and 22 are deployed to cause the contractile fibers in repopulated zone 15 to induce a contraction in a direction that aids hemodynamic function. A current flows from one electrode 20 or 22 to the other, generally by ionic conduction within the tissue, rather than by cell-to-cell conduction mediated by gap junctions.

Transplanted contractile biological material tends to orient itself in the direction in which the tissue stretches. Accordingly, the contractile fibers of the transplanted material generally will, with time, align with nearby cardiac muscle fibers.

It is not necessary to the invention that all transplanted biological material contributes to contraction. Undifferentiated cells, for example, may undergo differentiation in response to stimulation, and may develop contractile capability. Also, some transplanted biological material may support the contractile biological material. Endothelial cells, for example, may promote vascularization in repopulated zone 15, and genetic material may promote differentiation or phenotypic conversion of other cells.

Electrodes 20 and 22 are coupled via leads 24 and 26 to an implantable medical device (IMD) (not shown in FIG. 1), such as a pacemaker, pacemaker/defibrillator, medical monitor or the like. The IMD generates one or more electrical stimuli, which are delivered to the epicardium via electrodes 20 and 22.

In FIG. 1, pacing and sensing leads are deployed in the chambers of heart 10 to monitor heart 10 and to administer pacing, defibrillation, cardiac potentiation or other therapies to heart 10. The pacing and sensing leads may be coupled to the same IMD as leads 24 and 26, or may be coupled to a different IMD. For purposes of simplicity, it will be assumed that the pacing and sensing leads are coupled to the same IMD as leads 24 and 26.

An atrial lead 28 extends from the IMD through the superior vena cava 30 and into the right atrium 32. The distal end of atrial lead 28 includes one or more pace/sense electrodes 34. A ventricular lead 36 extends from the IMD through superior vena cava 30, through right atrium 32 and into the right ventricle 38. The distal end of ventricular lead 36 includes one or more pace/sense electrodes 40. Electrodes 34 and 40 may be bipolar or unipolar. Although shown in FIG. 1 as deployed inside the chambers of heart 10, the leads may be deployed epicardially, endocardially, intravascularly or in any combination thereof.

In the example depicted in FIG. 1, the IMD is configured to generate pacing stimulations, which are delivered to right atrium 32 or right ventricle 38 via pace/sense electrodes 34 and 40. In addition, the IMD senses electrical activity in heart 10 via electrodes 34 and 40. In particular, the IMD detects atrial and ventricular activations via electrodes 34 and 40. Electrodes 34 and 40 may be coupled to sense amplifiers that detect whether electrical activity exceeds a sensing threshold. In this way, the IMD detects P-waves indicative of atrial activation and R-waves indicative of ventricular activation. The IMD may also detect T-waves via electrodes 34 and 40, which indicate ventricular repolarization that occurs at the completion of ventricular contraction.

In the example depicted in FIG. 1, the IMD is further configured to generate stimulations to right atrium 32 or right ventricle 38 via pace/sense electrodes 34 and 40 as part of cardiac potentiation therapy (CPT). As discussed in more detail below, CPT includes delivering timed electrical stimuli to the heart to further improve diastolic filing and cardiac output.

The IMD may also apply digital signal analysis to signals sensed via electrodes 34 and 40. The signals may be amplified and converted to multi-bit digital signals by an analog-to-digital (A/D) converter. A microprocessor may employ digital signal analysis techniques for various purposes, such as to classify the patient's heart rhythm or to analyze the morphology of the signals. During digital signal analysis, various cardiac parameters may be measured, such as the duration of the QRS complex and the Q-T interval.

Atrial lead 28 or ventricular lead 36 or both may include a defibrillation electrode to deliver defibrillation therapy under the control of the IMD. Defibrillation electrodes are desirable because patients who receive stimulations to repopulated zone 15 are at risk of fibrillation. Defibrillation electrodes provide added safety in light of this risk. Atrial and ventricular leads 28, 36 may also include other sensors, such as sensors that respond to the blood pressure inside heart 10.

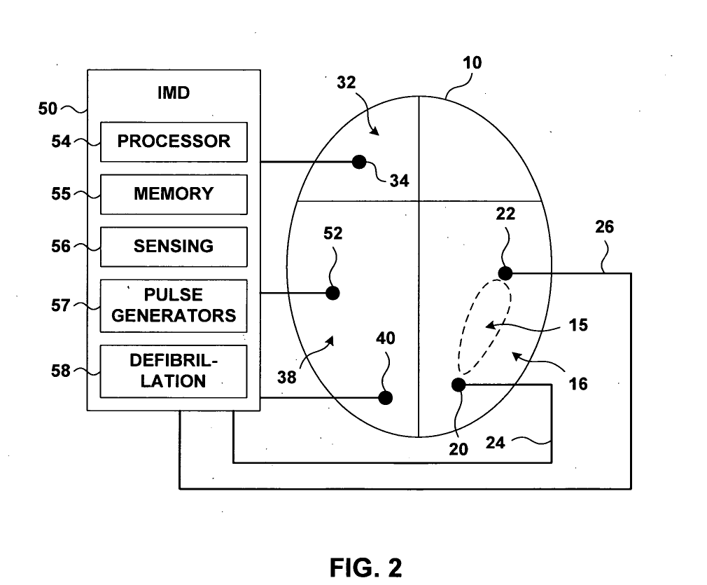

FIG. 2 is a symbolic diagram of heart 10 with an IMD 50. IMD 50 controls delivery of electrical stimulation to repopulated zone 15 via electrodes 20 and 22. IMD 50 further senses atrial and ventricular activity via electrodes 34 and 40, and may also deliver pacing therapy to heart 10 via electrodes 34 and 40. In addition, IMD 50 receives signals from a sensor 52. Sensor 52 may be any sensor that detects any signal reflecting physiological activity. In general, sensor 52 may be selected to detect the stage of the cardiac cycle of heart 10. Sensor 52 may be, for example, an electrode disposed on the epicardium, or a pressure sensor deployed inside right ventricle 38, or a sound sensor deployed at any site in the body where heart sounds can be detected. Sensor 52 may also be, for example, a lead tip accelerometer that senses the wall motion of heart 10 in one, two or three dimensions. Sensor 52 may be, but need not be, deployed on ventricular lead 36 shown in FIG. 1. Sensor 52 may also include a plurality of sensors, such as intracardiac impedance sensors that detect changes in impedance that occur during the cardiac cycle.

IMD 50 includes at least one processor 54 that regulates delivery of electrical stimulation to repopulated zone 15, and that further supervises pacing and defibrillation operations. Processor 54 comprises, for example, any microprocessor, digital signal processor, application specific integrated circuit or full custom integrated circuit.

Processor 54 determines whether heart 10 is in the ejection phase of the cardiac cycle, and causes electrical stimulation to be delivered to repopulated zone 15 during the ejection phase. Processor 54 may, for example, store a signal from sensor 52 in memory 55 and analyze the stored signal. Processor 54 may analyze a pressure signal, for example, to identify an occurrence at which a maximum change of sensed pressure in the ventricle occurs during a cardiac cycle. This analysis technique will be described in more detail below. Memory 55 may comprise any combination of volatile and non-volatile memory.

Processor 54 may also analyze electrical signals received via electrodes 20, 22, 34 or 40 and sensed via a sensing module 56 such as a peak sense and threshold measurement circuit. In particular, processor 54 can use the sensed electrical signals to determine whether heart 10 is in the ejection phase.

In addition, processor 54 controls delivery of CPT, as described below. In particular, processor 54 controls the timing of delivery of CPT stimulations to improve cardiac output. CPT stimulations can improve cardiac output in two respects. CPT enhances relaxation of heart 10 during diastole, thereby improving diastolic filling. CPT also causes heart 10 to produce more forceful contractions during systole. The result is increased stroke volume and increased cardiac output.

IMD 50 includes one or more pulse generators 57 to deliver stimulations under the control of processor 54. Pulse generators 57 generate pulses that are delivered to repopulated zone 15, and may also serve as pacer output circuitry, generating pacing pulses to be delivered to right atrium 32 and right ventricle 38 by electrodes 34 and 40, under the control of processor 54. Processor 54 may use pacing pulses from pulse generators 57 or sensed P-waves, QRS complexes or T-waves, or any combination thereof, to determine whether heart 10 is in the ejection phase, which may be useful for timing delivery of pulses to repopulated zone 15. Pulse generators 57 further generate CPT stimulations that are delivered to right atrium 32 and right ventricle 38 by electrodes 34 and 40, under the control of processor 54.

IMD 50 further includes a defibrillation module 58 delivers defibrillation or cardioversion therapy under the control of processor 54. Defibrillation module 58 delivers high-energy shocks to heart 10 when heart 10 exhibits a dangerous arrhythmia. As noted above, defibrillation capability is not necessary to the invention, but is desirable. The occurrence of an acute myocardial infarction is often associated with sudden cardiac death caused by ventricular arrhythmias, and the ventricular arrhythmias can be effectively terminated by defibrillation therapy.

In one embodiment of the invention, IMD 50 controls the rhythm of heart 10 by administering pacing stimulations via atrial electrode 34 and ventricular electrode 40. IMD 50 further administers stimuli proximate to repopulated zone 15 via epicardial electrodes 20 and 22. IMD administers stimuli via electrodes 20 and 22 to coincide with the pumping action of heart 10. More specifically, IMD 50 administers stimuli via electrodes 20 and 22 when the pulmonary and aortic valves of heart 10 are open. IMD 50 further administers CPT stimulations to improve the pumping action of heart 10.

The stimuli administered to repopulated zone 15 via electrodes 20 and 22 can cause tissue in or proximate to repopulated zone 15 to contract in synchrony with other cardiac tissue. In particular, the stimuli cause the biological material transplanted in and proximate to repopulated zone 15 to contract. The stimuli generally do not cause scar tissue in infarct region 14 to contract.

The response of various biological materials to stimulation is currently a subject of research. There is evidence that electrical stimulation of some kinds of biological material can cause the biological material to assume characteristics of muscle tissue. The biological material may, for example, show signs of differentiation, or may exhibit indications of phenotypic conversion, such as increased numbers of mitochondria, greater fatigue resistance or enhanced contractile properties. Some biological material, after repeated stimulation, begins to take on characteristics of muscle, such as skeletal muscle. It is believed possible that electrical stimulation of biological material may cause differentiation into cardiac muscle, which couples to the host tissue. In other words, ongoing research may include supplanting scar tissue with living contractile tissue. In addition, electrical stimulation may promote proliferation of the transplanted cells, thereby repopulating infarct region 14 with contractile tissue.

For purposes of describing the invention, however, it is assumed that at least a portion of the transplanted biological material contracts in some fashion in response to electrical stimulation from electrodes 20 and 22. It is not necessary that the biological material supplant scar tissue. It is not necessary for the invention that all transplanted biological material be contractile, or that the transplanted biological material be contractile upon transplantation. Rather, some transplanted biological material may be non-contractile when implanted, and may become contractile or conductive at the transplant site in response to stimulation. Also, it is not necessary for the invention that the transplanted biological material assume any particular characteristics or phenotype.

As noted above, the invention is not limited to any particular biological material or materials. For purposes of illustrating the invention, it will be assumed that the transplanted biological material in and proximate to infarct region 14 has characteristics of skeletal muscle. In other words, the biological material contracts in response to electrical stimulation, but need not contract in the same way as cardiac muscle. In general, skeletal muscle contracts and relaxes more rapidly than cardiac muscle. Skeletal muscle contracts and relaxes within ten to thirty milliseconds, but cardiac muscle contracts and relaxes within about a hundred milliseconds. Accordingly, IMD 50 delivers a set of stimuli to the biological material to cause the biological material to contract and relax in a manner similar to cardiac muscle. In other words, IMD 50 delivers a set of stimuli to the biological material to cause the biological material to contribute to the pumping action of heart 10.

The set of stimuli is delivered at a time in the cardiac cycle when contraction of the biological material contributes to hemodynamic function. In the example depicted in FIGS. 1 and 2, in which infarct region 14 and repopulated zone 15 are on the left ventricle, IMD 50 delivers the stimuli to coincide with the ventricular activation and pumping. The duration, timing and other characteristics of the set of stimuli depend upon the location of the biological material.

CPT stimulations further improve the pumping efficiency of heart 10 by inducing post-extrasystolic potentiation, which comprises managing the distribution of calcium ions that contribute to contraction of cardiac myocytes. At the molecular level, a contraction occurs when a myocin molecule binds to and pulls an actin filament. Calcium ions, or Ca²⁺, make such bindings possible by binding to troponin molecules on the actin filaments.

An action potential, which triggers a contraction, also triggers release of Ca²⁺ from the sarcoplasmic reticulum, which surrounds the cardiac myofibrils, into the cytosol. The Ca²⁺ in the cytosol is free to engage with the troponin. After the myocin pulls the actin, the Ca²⁺ disengages from the troponin, is taken up by the sarcoplasmic reticulum, and the myocytes relax. The sarcoplasmic reticulum takes up Ca²⁺ actively, using a sarcoplasmic reticulum Ca²⁺-adenosinetriphosphatase (SERCA2) pump, also called a Ca²⁺-ATPase pump. The pump is an energy-consuming pump that actively transports Ca²⁺ from the cytosol to the sarcoplasmic reticulum.

CPT stimulations cause the pump to be activated for a longer time than would naturally be the case. Following a ventricular contraction, the muscles of the ventricle repolarize. On an electrocardiogram, the repolarization manifests itself as a T-wave. A CPT stimulation applied to the ventricles during or shortly after repolarization will ordinarily not induce the ventricles to contract and pump blood. The CPT stimulation will, however, reactivate the pump to take up additional Ca²⁺ into the sarcoplasmic reticulum. As a result of the CPT stimulation, more Ca²⁺ is taken up by the sarcoplasmic reticulum than would be taken up without CPT stimulation, and the concentration of Ca²⁺ in the sarcoplasmic reticulum is increased. Because the concentration of Ca²⁺ in the sarcoplasmic reticulum is increased, the concentration of Ca²⁺ in the cytosol is decreased, resulting in more relaxation of the cardiac muscles, which in turn increases cardiac filling during diastole.

When an action potential triggers a subsequent contraction, the same action potential triggers release of Ca²⁺ from the sarcoplasmic reticulum. Because the concentration of Ca²⁺ in the sarcoplasmic reticulum has been increased by CPT during diastole, there is an increase in the concentration of Ca²⁺ released during systole. In other words, an action potential triggers release of a large bolus of Ca²⁺ into the cytosol. Increased concentration of Ca²⁺ in the cytosol results in increased binding of actin and myocin molecules during systole, resulting in a more forceful contraction.

In other words, CPT stimulations affect the concentration of calcium ions that regulate contraction of cardiac myocytes. CPT causes more Ca²⁺ to be drawn from the cytosol during diastole, and causes a larger bolus of Ca²⁺ to be released from the sarcoplasmic reticulum during systole. These effects result in enhanced cardiac filling during diastole and more forceful contractions during systole, thereby increasing the stroke volume of heart 10. In this way, CPT stimulations cause the heart to produce a greater cardiac output at higher systolic and lower diastolic pressures.

As noted above, a CPT stimulation applied to the ventricles during or shortly after repolarization will ordinarily not induce the ventricles to pump blood. In general, CPT stimulations comprise applying one or more extrasystolic electrical stimuli to one or more heart chamber at a time when application of the stimulations would not result in a sizeable contraction. In a typical heart, a stimulation delivered less than 250 milliseconds following an action potential will not result in another contraction, because the heart will still be in its refractory period and not ready to contract. CPT stimulations generally will, however, induce post-extrasystolic potentiation by drawing more Ca²⁺ from the cytosol into the sarcoplasmic reticulum, as described previously.

A patient that has suffered a myocardial infarction can benefit from stimulations to repopulated zone 15 in concert with CPT stimulations. A patient that has suffered a myocardial infarction may have lost hemodynamic function, and the stimulations can compensate for that loss of function. Stimulations applied to infarct region 14 and repopulated zone 15 may contribute to hemodynamic function as described above, by influencing transplanted contractile biological material to orient itself with native tissue and contribute to pumping. CPT stimulations may further contribute to hemodynamic function by enhancing cardiac filling and improving the forcefulness of the cardiac contractions.

Although CPT stimulations principally enhance the performance of native cells, it is possible for CPT also to provide a measure of guidance to the transplanted biological material proximate to infarct region 14. Some kinds of transplanted biological material, such as cardiac myocytes, may respond to CPT more favorably than other kinds of transplanted biological material. It is not necessary to the invention, however, that the transplanted biological material respond to CPT stimulations.

Nor is it necessary to the invention that stimuli be delivered proximate to repopulated zone 15 on every cardiac cycle and that CPT stimulations also be delivered on every cardiac cycle. The invention supports applications in which stimuli are delivered proximate to repopulated zone 15 at some times, and CPT stimulations are delivered at other times. The invention also supports applications in which CPT stimulations continue while stimuli delivered proximate to repopulated zone 15 are suspended, in order to reduce the load on repopulated zone 15 and reduce fatigue of the maturing tissue.

Over time, as the contractile fibers of the transplanted biological material mature and become more aligned with native cardiac muscle fibers, stimuli delivered proximate to repopulated zone 15 via epicardial electrodes 20 and 22 may taper off, while CPT stimulations may hold steady or increase. Continuing CPT stimulations help heart 10 pump blood more efficiently by managing the distribution of calcium ions in the sacroplasmic reticulum and cytosol.

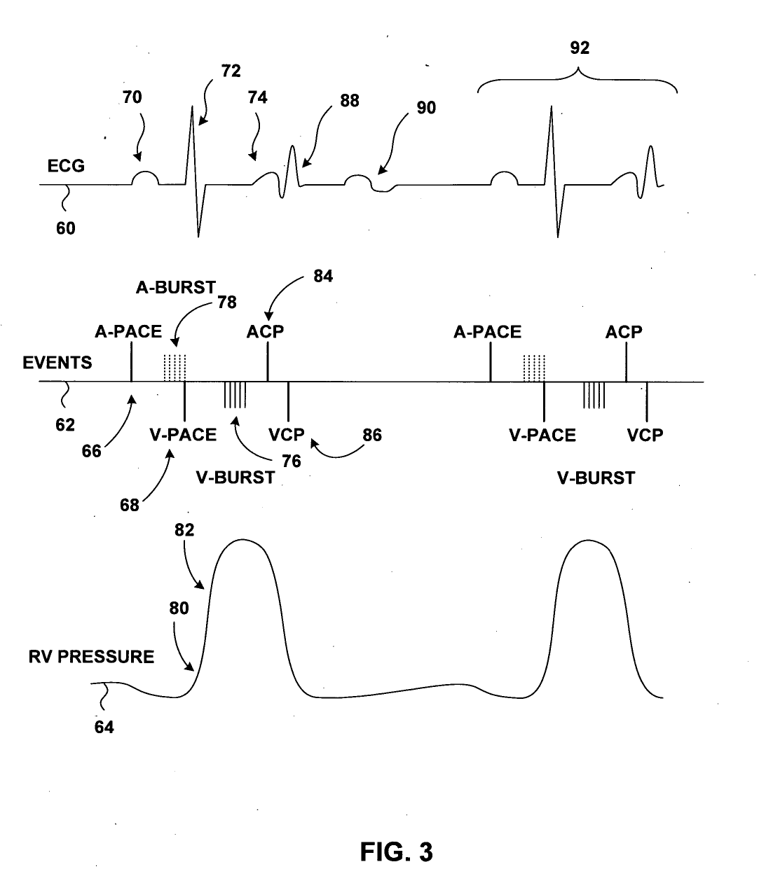

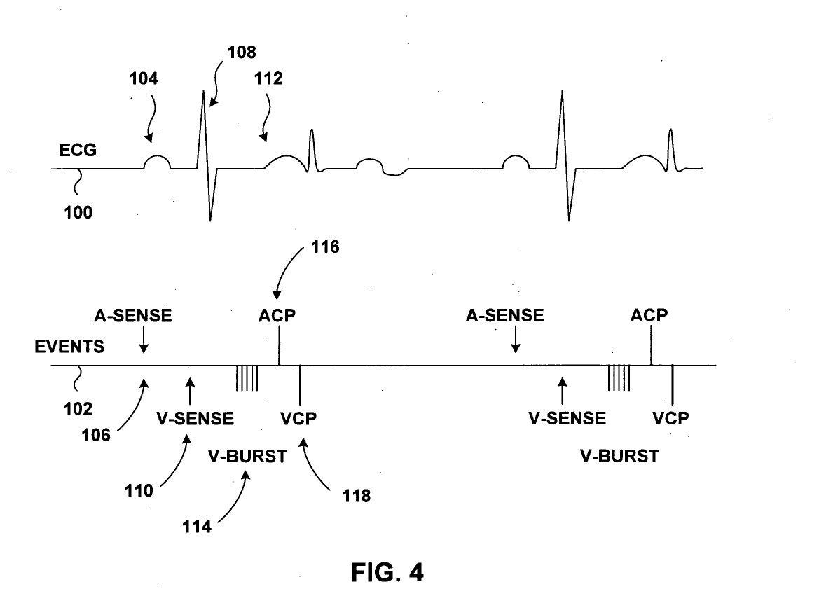

FIGS. 3 and 4 are timing diagrams illustrating techniques for delivery of stimuli to the transplanted biological material, with CPT. FIG. 3 depicts timing of stimuli to the transplanted biological material and CPT in conjunction with pacing stimuli delivered by IMD 50, and FIG. 4 depicts timing of stimuli to the transplanted biological material and CPT in conjunction with sensed cardiac events.

FIG. 3 includes three signals. An electrocardiogram (ECG) 60 shows the electrical activity of heart 10. ECG 50 may be sensed with electrodes deployed on the body of the patient, including electrodes deployed epicardially or endocardially. An event marker 62 shows electrical stimulations delivered under the control of IMD 50. A pressure waveform 64 shows pressure inside a ventricle of heart 10, which may be measured by a pressure sensor deployed in right ventricle 38.

As depicted in FIG. 3, IMD 50 delivers an atrial pacing pulse, identified as “A-Pace” 66, and a ventricular pacing pulse, identified as “V-Pace” 68. In the example shown in FIGS. 1 and 2, electrodes 34 and 40 deliver A-Pace 66 and V-Pace 68.

In response to delivery of A-Pace 66, the atria of heart 10 depolarize. The depolarization manifests as a P-wave 70 in ECG 60. Delivery of V-Pace 68 causes the ventricles of heart 10 to depolarize, which manifests in ECG 60 as the QRS complex 72. Repolarization of the ventricles manifests in ECG 60 as T-wave 74, but as discussed below, T-wave 74 does not resemble a conventional T-wave because of CPT stimulations.

IMD 50 delivers a set of stimuli via epicardial electrodes 20 and 22, identified as “V-Burst” 76, to the biological material. In general, V-Burst 76 comprises a series of distinct stimulations. The amplitude, pulse width, number of stimulations and interval between stimulations may vary as a function of the biological material stimulated and the response. These stimulation parameters may be adjusted for a particular patient, e.g., to enhance stroke volume or cardiac synchrony. V-Burst 76 should typically deliver sufficient energy to excite the tissue, but not so much energy as to unnecessarily drain the power supply or damage the tissue. Although the discussion below will focus upon delivery of V-Burst 76, the invention encompasses embodiments in which biological material that has been transplanted onto an atrium is stimulated with a set of stimuli, identified as “A-Burst” 78, to aid the pumping function of the atria.

IMD 50 delivers V-Burst 76 at a time when contraction of transplanted cells in repopulated zone 15 will assist in hemodynamic function. In addition, IMD 50 avoids delivering V-Burst 76 at a time when heart 10 is vulnerable to induction of arrhythmias. In the example shown in FIGS. 1 and 2, V-Burst 76 may aid the pumping function during contraction of the ventricles, and in particular, when the ejection phase begins and the aortic and pulmonary valves are open.

Delivery of V-Burst 76 at other times in the cardiac cycle would provide lesser hemodynamic assistance, or no hemodynamic assistance at all. Delivery of V-Burst 76 prior to QRS complex 72 would not assist in hemodynamic function, because the ventricles would be resting rather than contracting. Delivery of V-Burst 76 during the isovolumetric contraction phase would generally provide little hemodynamic assistance, and may cause the biological material to become fatigued. Stimulating the biological material during repolarization would not only fail to aid hemodynamic function, but may generate a dangerous arrhythmia such as ventricular fibrillation. Accordingly, IMD 50 times the delivery of V-Burst 76 to take place when the aortic or pulmonary valves are open. In general, the valves are open during a portion of the S-T segment, i.e., at some time between the end of the QRS complex 72 and T-wave 74.

The invention encompasses timing the delivery of V-Burst 76 occur during the ejection phase, i.e., while the aortic and pulmonary valves are open and blood is being ejected from the ventricles. Various techniques exist for delivering V-Burst 76 at a time when heart 10 is in the ejection phase.

One technique is to deliver V-Burst 76 at an interval following an intrinsic cardiac event, or following an event under the control of IMD 50. For example, IMD 50 may deliver V-Burst 76 at time interval after the R-wave, which coincides with QRS complex 72, or at a time interval following delivery of a ventricular pace 68. The time interval can be a function of several factors, such as the heart rate of the patient, or other factors that affect the S-T segment.

Another timing technique uses the pressure inside a ventricle as an indicator of whether a valve is closed or open. A pressure sensor may be deployed in right ventricle 38 or in left ventricle 16. In FIG. 3, it is assumed that a pressure sensor has been deployed in right ventricle 38. Pressure signal 64 reflects the sensed pressure.

In a cardiac cycle, ventricular depolarization causes ventricular contraction. For a short period, no blood leaves the ventricles, and the contraction of the ventricles is isovolumetric. During isovolumetric contraction, the pressure in the ventricles builds, but is insufficient to force blood through the pulmonary or the aortic valve. On pressure signal 64, the onset of isovolumetric contraction is reflected in a sharp upturn 80 of pressure signal 64.

When the pressure in right ventricle 38 overcomes the pressure in the pulmonary arteries, the blood drives the pulmonary valve open, and right ventricle 38 ejects blood into the pulmonary arteries. When the pulmonary valve opens, contraction is no longer isovolumetric. Pressure in right ventricle 38, although still increasing due to ventricular contraction, increases at a slower rate. As a result, there is an inflection point 82 in right ventricular pressure signal 64 when the pulmonary valve opens. Inflection point 82 represents the point of maximum change of pressure with time. In right ventricular pressure signal 64, inflection point 82 is the point of maximum slope.

Inflection point 82 may be found by analysis of pressure signal 64. For example, IMD 50 may find the maximum value of the first derivative of pressure signal 64, or a corresponding zero crossing in the second derivative of pressure signal 64. By sensing the inflection point or the maximum change in pressure, the time of ejection from right ventricle 38 can be identified.

A similar process occurs in left ventricle 16, and a signal from a pressure sensor in left ventricle 16 may be analyzed in a similar fashion to determine the time that the pressure forces open the aortic valve. For many patients, deployment of a pressure sensor in right ventricle 38 can adequately identify the opening of both the pulmonary and aortic valves, because both valves typically open at about the same time.

In this way, by identifying an occurrence at which a maximum change of sensed pressure in a ventricle occurs, IMD 50 can detect when heart 10 enters the ejection phase. IMD 50 delivers V-Burst 76 during the ejection phase.

In a typical embodiment, IMD 50 need not analyze a pressure sensor signal with every cardiac cycle. Instead, IMD 50 may deliver V-Burst 76 at an interval following an intrinsic or paced cardiac event, and may perform pressure signal analysis from time to time to determine whether the interval causes stimulation to take place during the ejection phase.

To improve cardiac output on the subsequent cardiac cycle, IMD 50 delivers CPT stimulations to the atria, the ventricles, or both. IMD 50 delivers an atrial coupled pace (ACP) 84 to right atrium 32 via electrode 34. ACP 84 is “coupled” to an atrial event, in that timing of delivery of ACP 84 depends upon the timing of the atrial event. In the example of FIG. 3, the atrial event is A-Pace 66. IMD 10 also delivers a ventricular coupled pace (VCP) 86 to right ventricle 38 via electrode 40. VCP 86 is coupled to a ventricular event, which in the example of FIG. 3 is V-Pace 68. As illustrated in FIG. 3, the time interval between A-Pace 66 and ACP 84 need not be the same as the time interval between V-Pace 68 and VCP 86. Although depicted in FIG. 3 as single pulse stimulations, ACP 84, VCP 86 or both may also comprise multiple pulse stimulations.

ACP 84 and VCP 86 are delivered when the heart is incompletely prepared to contract as part of a new cardiac cycle. As a result, the stimulations activate heart 10 but do not contribute to pumping of blood. There is very little mechanical motion of the heart muscle in response to the stimulations, and there is insufficient contraction to open the valves and eject blood. Even so, delivery of VCP 86 may generate an R′-wave 88 on ECG 60, as the stimulation traverses the ventricles. VCP 86 may also result in a T′-wave 90 on ECG 60, as the ventricles repolarize in response to VCP 86.

As discussed above, CPT stimulations excite the pumps that actively transport Ca²⁺ to the sarcoplasmic reticulum, causing the pumps to be activated for a longer time than would naturally occur. As a result of the CPT stimulations, more Ca²⁺ is taken up by the sarcoplasmic reticulum than would be taken up without CPT stimulation, and the concentration of Ca²⁺ in the sarcoplasmic reticulum is increased, and the heart relaxes to a greater degree. On a subsequent cardiac cycle 92, an action potential triggers release of an increased concentration of Ca²⁺ from the sarcoplasmic reticulum, resulting heart 10 having a stronger contraction on subsequent cardiac cycle 92.

FIG. 4 is a timing diagram similar to FIG. 3, showing an ECG 100 and an event marker 102. For purposes of simplicity, the pressure signal is omitted from FIG. 4. FIG. 4 also omits delivery of an A-Burst for simplicity.

In the example of FIG. 4, cardiac events are sensed, rather than paced. In particular, FIG. 4 depicts timing of stimuli to the transplanted biological material and CPT in conjunction with sensed, rather than paced, cardiac events. When the atria of the heart depolarize, as manifested by P-wave 104, IMD 50 senses the depolarization, as indicated by A-Sense 106. When the ventricles of the heart depolarize, as manifested by QRS complex 108, IMD 50 senses this event, as indicated by V-Sense 110. Repolarization of the ventricles manifests in ECG 100 as T-wave 112.

IMD 50 delivers a set of stimuli via epicardial electrodes 20 and 22, identified as “V-Burst” 114, to the transplanted biological material. Delivery of V-Burst 114 may be similar to delivery of V-Burst 76 in FIG. 3. Timing for V-Burst 114 can be coupled to electrically sensed events such as V-Sense 110, or to pressure measurements, as described above. In general, IMD 50 times the delivery of V-Burst 114 to occur during the ejection phase to cause the transplanted biological material to contribute to hemodynamic function.

IMD 50 delivers CPT stimulations to the atria, the ventricles, or both. In FIG. 4, IMD 50 delivers an ACP 116 to right atrium 32. Timing of ACP 116 can be coupled to a sensed event, such as A-Sense 106 or V-Sense 110. Similarly, IMD 50 delivers VCP 118 to right ventricle 38, coupled to a sensed event. When ACP 116 or VCP 118 is coupled to a sensed event, the timing need not be the same as when the coupling is to a paced event, as depicted in FIG. 3. As described above, the CPT stimulations affect the distribution of calcium ions, resulting in enhanced relaxation during diastole and more forceful ejection during systole.

The invention is not limited to timing stimulations of transplanted biological material as a function of intrinsic cardiac events, paced cardiac events, or pressures. Other sensors and signals can be used to detect the opening of a pulmonary or aortic valve, or to estimate reliably when the valves are open. An accelerometer, a flow meter, an intracardiac impedance sensor or a sonomicrometer, for example, may generate a signal that can be used to detect whether the heart is in the ejection phase. A microphone that detects heart sounds also may detect the onset of isovolumetric contraction by detecting the closure of the atrioventricular valves. Identifying the onset of isovolumetric contraction may be used for accurately estimating when heart 10 is in the ejection phase. Similarly, the invention is not limited to timing CPT stimulations as a function of intrinsic cardiac events or paced cardiac events or combinations thereof.

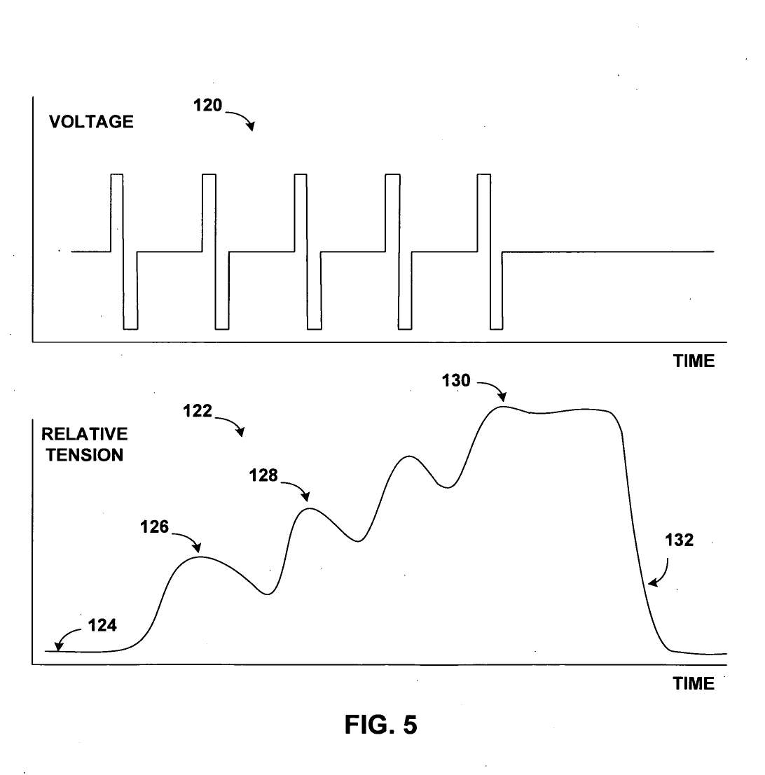

FIG. 5 illustrates an exemplary V-Burst 120 comprising five bipolar pulses having square wave shapes. Each pulse has an amplitude above the threshold potential of the contractile material in repopulated zone 15. The amplitude of the voltage depends upon the number of contractile fibers affected, which depends upon the distance between stimulating electrodes 20 and 22. For example, the amplitude of the voltage can be about one volt per millimeter of separation between electrodes 20 and 22. V-Burst 120 may include one pulse every one hundredth of a second, and each pulse may have a pulse width of a millisecond. Generally speaking, the shape of the waveform is not as important as the energy it provides to the tissue, because the stimuli ought to be strong enough to excite the newly formed contractile tissue. Also, the stimulation waveform should typically be charge-balanced, meaning that residual positive and negative charges cancel following each pulse. Charge-balancing reduces electrode corrosion and prevents harm to the tissue surrounding the electrodes. It is not necessary, however, that the positive and negative segments of the pulse have the same shape or duration.

In an application in which the biological material includes skeletal muscle, the pulses of exemplary V-Burst 120 come one after another, and do not allow the muscle to relax fully after each pulse. A graph of contractile activity 122 shows that, upon stimulation, muscle tension increases from a relaxed state 124 to a peak 126, and then begins to decline. Before the muscle can relax fully, however, another stimulating pulse causes a summation response 128, increasing the tension further or maintaining the tension. Additional stimulating pulses can cause a sustained tetanic contraction 130. When the stimulation ends, the tension returns 132 to a resting state.

The effect of stimulating the biological material with a set of stimuli is to cause the skeletal muscle cells to contract for a longer time than skeletal muscle cells would ordinarily contract. In other words, the effect is to cause skeletal muscle cells to have a contraction time comparable to that of cardiac muscle cells. In the time shortly after transplantation of the biological material, stimulation therapy can be suspended for some cardiac cycles to allow the tissue to recover and to build a tolerance to fatigue. For example, the stimulation may be delivered on every fifth cardiac cycle shortly after transplantation, with the frequency of stimulation increasing over time.

In addition, stimulation therapy can be suspended from time to time so that IMD 50 or another device can monitor the hemodynamic function of heart 10. IMD 50 or another device may monitor, for example, hemodynamic parameters such as cardiac output, stroke volume, ventricular pressure, blood flow rate and the like. By such monitoring, IMD 50 or another device can gather data that indicate whether or not the transplanted tissue is contributing to hemodynamic function when stimulated. The data may indicate, for example, that the transplanted biological material is ineffective in contributing to hemodynamic operation, in which case stimulation therapy may be discontinued to conserve power for other functions, such as defibrillation or pacing. When the transplanted biological material is ineffective in contributing to hemodynamic operation, physician intervention, such as intervention to transplant new biological material, may also be indicated. The data may also indicate that or that the biological material has become integrated with the native myocardium, in which case stimulation therapy may be reduced to allow the new tissue to be excited intrinsically via the endogenous conduction system of heart 10. Stimulation therapy may be reduced by delivering electrical stimulation at a reduced per-cardiac-cycle frequency, such as by delivering one set of stimulating pulses for every five cardiac cycles instead of one set of stimulating pulses for every cardiac cycle. The data may also indicate that continued stimulation therapy is appropriate.

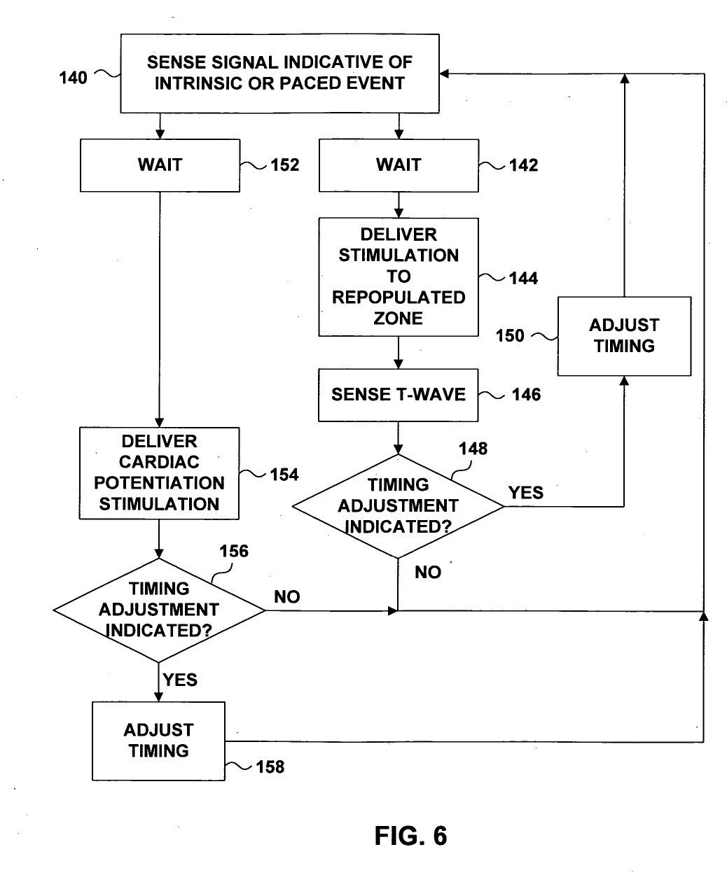

FIG. 6 is a flow diagram illustrating an embodiment of the invention. In the embodiment depicted in FIG. 6, stimulation of the repopulated zone and CPT stimulation follow parallel paths. IMD 50 senses one or more signals indicative of an intrinsic or paced cardiac event (140). The sensed signal may include a biological signal, such as a pressure signal, a signal responsive to motion, an electrical signal or a heart sound signal. The signal may also correspond to a pacing event, such as the delivery of a ventricular pace. Depending upon the sensed signal, IMD 50 may wait for a time interval (142) for heart 10 to enter the ejection phase, and then IMD 50 delivers stimulation to the repopulated zone (144). IMD 50 times the delivery of stimulation (144) to coincide with the ejection phase of heart 10.

In one embodiment of the invention, the waiting interval associated with stimulation of the repopulated zone (142) may be eliminated. When the signal indicative of a cardiac event is a ventricular pressure signal, for example, IMD 50 may deliver stimulation (144) promptly upon sensing the maximum change of pressure, with no waiting. Analysis of a pressure signal with every cardiac cycle may result in signal processing that drains the power supply for IMD 50, however.

In a typical embodiment, IMD 50 senses at least one signal indicative of a cardiac event on each cardiac cycle, and senses other signals less frequently. In an illustrative application, IMD 50 senses the R-wave sense amplifiers on every cardiac cycle (140), waits for a time interval after the R-wave (142), and delivers a stimulation at a time when heart 10 is expected to be in the ejection phase (144). When sensing an R-wave with a sense amplifier consumes less power than pressure signal analysis, it can be more efficient to time the delivery of stimulations with respect to the sensed R-wave. Pressure signal analysis may still be performed periodically to assure that stimulation is taking place during the ejection phase, but pressure signal analysis need not be performed on every cardiac cycle.

IMD 50 may further sense the T-wave (146). As noted above, the stimulation associated with excitation of the repopulated zone should generally take place while the aortic and pulmonary valves are open, and the valves are generally closed by the time the T-wave occurs. By monitoring the T-wave (146), IMD 50 verifies that the stimulation of the repopulated zone (144) takes place during the S-T segment, and that the stimulation does not take place when heart 10 is vulnerable to induction of arrhythmias. The T-wave may be, but need not be, monitored on every cardiac cycle.

From time to time, IMD 50 may determine whether the stimulations to the repopulated zone are being delivered at an appropriate time (148). Such a determination may be based upon signals such as pressure signals and T-wave monitoring signals, which can indicate whether the waiting period after R-wave detection is appropriate, or too short or too long. When a timing adjustment is needed, IMD 50 increases or decreases the waiting period (150). IMD 50 may check the timing (148) periodically, such as after a fixed number of cardiac cycles, or in response to an event, such as an increase in heart rate, or both.

Sensing an intrinsic or paced signal indicative of a cardiac event (140) can also be used to time delivery of CPT stimulations. The signal that is used to time delivery of CPT stimulations may be, but need not be, the same as the signal used to time delivery of stimulations to the repopulated zone. For example, stimulations to the repopulated zone may be coupled to a pressure signal, while CPT stimulations are coupled to sensing of an R-wave.

After a waiting period (152), IMD 50 delivers CPT stimulations (154). IMD 50 may apply a first waiting period before delivering an ACP, and a second waiting period before delivering a VCP. IMD 50 may further determine whether the CPT stimulations are being applied at appropriate times, and may adjust the waiting periods (158) when appropriate. As described above, CPT stimulations are generally more effective when delivered at a time when heart 10 is not ready to contract. A waiting period may be shortened when, for example, a CPT stimulation induces a contraction.

The various embodiments of the invention may result in one or more advantages. While necrotic tissue is less elastic than healthy cardiac tissue, transplantation of biological material can improve the elasticity of the heart. In addition, necrotic tissue adversely affects hemodynamic function, but transplantation of biological material, combined with electrical stimulation, can restore some pumping ability to a damaged region of heart tissue.

In addition, the invention can complement other treatments for myocardial infarction. Coronary artery bypass, for example, can address providing a blood supply to heart tissue, but does not address the effects of scar tissue upon the elasticity and the pumping ability of the heart. The invention, however, can address those concerns.

Further, use of CPT stimulations in concert with stimulation of transplanted biological material helps the heart recover some hemodynamic function that may have been lost due to myocardial infarction. CPT stimulations help the healthy heart tissues function more efficiently, thereby compensating to a degree for the loss of hemodynamic function due to necrotic tissues.

### EXAMPLE 1

The following example, which demonstrates some of the aspects of the invention, is for illustrative purposes. The subjects of the tests included nine canines.

Three canines formed the control group and six canines formed the “test” or “treatment” group. Skeletal muscle biopsies of approximately 5 grams were obtained from all animals from the masseter muscle for the isolation of skeletal muscle cells, or “satellite” cells. Details of a procedure to isolate and culture satellite cells are described in Chiu RC-J et al., “Cellular Cardiomyoplasty: Myocardial Regeneration With Satellite Cell Implantation,” Ann. Thorac. Surg. 60:12-8 (1995).

Two weeks after the biopsy procedure, myocardial infarction was induced in all animals by temporary occlusion of the left anterior descending (LAD) coronary artery followed by reperfusion. This technique is described in Kao R. L. et al., “Satellite Cell Transplantation to Repair Injured Myocardium,” Card. and Vasc. Regener. 1:31-42 (2000). Following the infarction/reperfusion, animals in the control group received injections of culture medium (Sigma), and animals in the treatment group received 5×10⁷ autologous satellite cells via intra-myocardial injection. Six weeks after the initial surgery, the animals were anesthetized, the chest was opened, and the instruments for physiologic measurement of the cardiovascular function were placed. Intravascular pressure catheters (Millar Instruments, Inc., Houston, Tex.) were advanced into the left ventricle and flow probes (Transonic System, Inc., Ithaca, N.Y.) were placed around the aorta.

All nine of the animals in the study were subjected to cardiovascular functional studies, during which the myocardium received electrical stimulation. The hemodynamic function of the heart of each animal was assessed before the animals received the electrical stimulation.

Unipolar epicardial leads were attached to the atrium and the ventricle to pace both chambers of the heart. Rib spreaders used in the surgery served as the return electrode for the unipolar pacing pulses sent to the atrium and the ventricle. Two more epicardial electrodes were attached to the myocardium, near the perimeter of the infarct region, which were used to deliver bipolar stimulation to the skeletal muscle formed in the infarct region. The epicardial electrodes were placed such that the electrical field created by these electrodes was perpendicular to the muscle fiber orientation, which allowed the capture of maximum number of fibers in the repopulated zone. All five leads, atrial and ventricular stimulation lead, return electrode connected to the rib spreader and the two leads going to the infarct zone were attached to a custom stimulator designed for the study.

The pacemaker portion of this custom stimulator provided stimulation pulses in DOO mode, meaning that both chambers were paced at all times, with no sensing of the intrinsic activation, and without any inhibition of the pacing. The pacing rate and the paced atrioventricular delay were chosen to be slightly higher than the intrinsic rate of the animal to overdrive the sinoatrial node and to assure that the pacemaker solely governed the timing of the atrial and ventricular contractions. The output amplitude of the stimulator was adjusted until capture of both chambers of the heart could be verified from the monitored surface ECG. Measured physiologic parameters, such as aortic flow and left ventricular pressure, while a DOO pacing was applied, formed the baseline for subsequent measurements.

Referring to FIG. 2, each animal received an atrial electrode 34 disposed in right atrium 32 and a ventricular electrode 40 disposed in right ventricle 38. Each animal further received stimulating electrodes 20 and 22 proximate to the region that had received the culture medium (in the case of the control group) or the satellite cells (in the case of the test group). Electrodes 20 and 22 were about 50 millimeters apart.

Referring to FIG. 3, each animal received an atrial pace 66, a ventricular pace 68 and a V-Burst 76. As noted above, the ECG 60 of each animal was monitored.

Because the satellite cells placed in or proximate to the infarct region were obtained from skeletal muscles having a fast twitch response, the stimulation included a train of five pulses in V-Burst stimulation 76. The duration of V-Burst stimulation 76 was 41 milliseconds. The object of delivering a set of pulses was to cause a long duration contraction of the skeletal muscle formed in the repopulated zone, which would augment the systolic function produced by the healthy native myocardium. The burst stimulation was applied in bipolar mode, i.e., between electrodes 20 and 22. The object of bipolar stimulation was to reduce the unintentional stimulation of the surrounding skeletal muscles of the chest.

Each pulse within the burst train was cathodic (negative) for one millisecond, anodic (positive) for eight milliseconds, and the stimulation circuitry was designed to remove all the charges left on the tissue during the cathodic stimulation by using the anodic pulse. Timing of the burst stimulation was determined using the ventricular stimulation pulse of the DOO pacer as a reference. Also, care was taken to adjust the timing of the burst stimulation to prevent the stimulation from coinciding with T-wave 74 and the vulnerable period of the cardiac cycle, to reduce the chance of inducing ventricular fibrillation. The amplitude of the pulses, and the delay between delivery of the ventricular pace and the burst train, were independently controlled.

Left ventricular blood pressure and aortic blood flow measurements were repeated during the application of the DOO pacing combined with burst stimulation, and were used to measure the added benefit from the stimulated contraction of the skeletal muscle in the repopulated zone. Mean arterial pressure was estimated as a function of diastolic and systolic pressures. Aortic output multiplied by mean arterial pressure yielded cardiac power, and cardiac power was indicative of hemodynamic function.

The data collected concerning hemodynamic function showed that hearts in the test group maintained an elastic structure, while the infarct regions of the hearts in the control group gained more plastic properties. The transplanted cells enhanced the elasticity of the heart, while the fibrous scar tissue in the control group did not.

When the amplitude of the pulses was at or above fifty volts (i.e., one volt per millimeter of electrode separation), and the delay between delivery of the ventricular pace and the burst train was about fifty milliseconds or longer, the power exhibited by the left ventricle showed considerable improvement, in comparison to the same animal when it was not stimulated with a burst train. Three out of six animals in the treatment group showed improvement in cardiac power when paced and stimulated with a burst train, with the improvement being about forty-five to ninety percent, while three did not show significant improvement. In the control group, none of the animals showed improvement in cardiac power.

Following the measurement of cardiac function, the animals were sacrificed, and the hearts were removed for morphological and histological examinations. The results of the examinations showed that animals in the treatment group developed healthy looking muscle tissue at the site of satellite cell implantation. In the control animals, by contrast, the infarct region had abundant connective tissue formed by fibrin and collagen, without evidence of cardiomyocytes.

The preceding example is illustrative of an application of the invention, in connection with delivery of stimulations to transplanted biological material. The invention is not limited to the particular test protocols described above.

### EXAMPLE 2

The following example demonstrates some further aspects of the invention for illustrative purposes. In a test that included nineteen study canines, heart failure was induced by either long duration rapid pacing or by shorter duration rapid pacing with ischemic infarction. The test animals were administered CPT stimulations, and cardiac output was measured. The test animals were studied in both conscious (resting and exercising) and anesthetized states, before and after heart failure.

In a control evaluation of animals with healthy hearts, CPT produced little change in cardiac output. Some control group animals demonstrated a decline in cardiac output and some demonstrated an increase, with only one animal demonstrating an increase of more than ten percent with CPT. In the heart failure test animals, however, changes in cardiac output after CPT were more pronounced. Only one heart failure animal failed to show an increase in cardiac output with CPT, and at least eight animals demonstrated increases in cardiac output of more than ten percent.

Further animal studies pertaining to CPT and considerations for stimulation timing are described in U.S. Pat. No. 6,738,667 and in U.S. Pat. App. No. 2004/0049235A1, which are incorporated herein by this reference.

The above experimental results suggest that an animal having heart failure could benefit from a combination of stimulation to transplanted biological material, with CPT. The stimulation to transplanted biological material can help produce healthy cells proximate to the infarct region that can contribute to hemodynamic function, and CPT stimulations can improve the hemodynamic performance of native tissues.

Moreover, the preceding specific embodiments are illustrative of the practice of the invention, and various modifications may be made without departing from the scope of the claims. For example, it is not necessary that a single device control pacing of the heart and CPT and delivery of stimulations to the repopulated zone. In one variation, a first device may be responsible for pacing and CPT, and another device may be responsible for delivering stimulations to the repopulated zone.

In another variation, the IMD may be a full-featured implantable device, providing a range of pacing therapies such as atrial, right ventricular, left ventricular and bi-ventricular pacing. A full-featured device may further provide therapies such as cardioversion therapy, defibrillation therapy and anti-tachycardia pacing in addition to CPT and stimulation of the repopulated zone. The invention encompasses all of these variations.

The electrode and sensor placements shown in FIGS. 1 and 2 are exemplary, but the invention encompasses other deployments as well. For example, the electrodes that stimulate the repopulated zone may be deployed endocardially. The electrodes may be deployed, for example, on the distal end of lead that enters the right ventricle, penetrates the septal wall, and fixes to the tissue in the left ventricle proximate to the infarct region. In another variation, the electrodes may be deployed in a vessel, via the coronary sinus and the great vein. Whether leads are deployed endocardially or via the coronary vasculature may depend upon whether the infarct region is accessible to such deployment. Some leads may include anticoagulation features.

The invention is not limited to any particular electrode placement. On the contrary, the stimulating electrodes would ordinarily be deployed according to the position and orientation of the infarct regain and repopulated zone of each individual patient. Although the examples in FIGS. 1 and 2 show damage to the left ventricle, the techniques of the invention may also be applied when there has been damage to the right ventricle, for example, or when there has been damage to the interventricular septum. Nor is the invention limited to any particular kind of electrodes or any particular technique or fixation mechanism for placing the electrodes. The electrodes may be, for example, small-surface-area electrodes or larger line electrodes sewn into the tissue, or electrodes deployed in patches applied to the tissue. The electrodes may include, for example, conventional metallic conductors or conductive polymers.

The invention is not limited to the particular schemes described herein for stimulation of the repopulated zone. Different biological material may respond differently to electrical stimulation. Accordingly, an IMD may be programmed to apply a stimulation scheme that works best for the patient. In addition, the invention does not exclude other stimulation therapies. For example, the invention includes subthreshold stimulation, which delivers insufficient energy to the biological material to cause contraction, but which may promote neovascularization of the repopulated zone or infarct region.

The invention may be embodied in a computer-readable medium with instructions that cause a programmable processor to carry out the techniques described above. A “computer-readable medium” includes but is not limited to read-only memory, Flash memory, EPROM and a magnetic or optical storage medium. The medium may comprise instructions for causing a programmable processor to electrically stimulate biological material transplanted in a myocardium of a heart during an ejection phase of a cardiac cycle, and apply CPT stimulations to improve hemodynamic performance. These and other embodiments are within the scope of the following claims.

1. A method comprising:
electrically stimulating biological material transplanted in a myocardium of a heart during an ejection phase of a cardiac cycle; and
electrically stimulating a chamber of the heart to induce post-extrasystolic potentiation.

2. The method of claim 1, wherein the biological material comprises cells selected from skeletal myoblasts, differentiated stem cells, undifferentiated stem cells, fibroblasts, endothelial cells and genetically engineered cells.

3. The method of claim 1, wherein the biological material comprises at least one of genes and a chemoattractant.

4. The method of claim 1, wherein electrically stimulating the biological material transplanted in the myocardium comprises electrically stimulating the biological material transplanted in an infarct region of the myocardium.

5. The method of claim 1, wherein electrically stimulating the biological material transplanted in the myocardium comprises electrically stimulating the biological material transplanted proximate to an infarct region of the myocardium.

6. The method of claim 1, further comprising:
sensing a biological signal; and
electrically stimulating the biological material in response to the biological signal.

7. The method of claim 6, wherein the biological signal is a first biological signal, the method further comprising:
sensing a second biological signal; and
electrically stimulating the chamber of the heart to induce post-extrasystolic potentiation in response to the second biological signal.

8. The method of claim 6, wherein the biological signal comprises at least one of a blood pressure signal, a cardiac depolarization signal, a cardiac repolarization signal, a cardiac impedance signal and a heart sound signal.

9. The method of claim 1, wherein electrically stimulating the biological material comprises delivering a set of stimulating pulses to the biological material.

10. The method of claim 1, wherein electrically stimulating the chamber of the heart to induce post-extrasystolic potentiation comprises:
delivering a first electrical stimulus to an atrium; and
delivering a second electrical stimulus to a ventricle.

11. The method of claim 1, wherein the cardiac cycle is a first cardiac cycle, the method further comprising:
suspending the electrical stimulation of the biological material during a second cardiac cycle; and
electrically stimulating the chamber of the heart to induce post-extrasystolic potentiation during the second cardiac cycle.

12. A computer-readable medium comprising instructions for causing a programmable processor to:
electrically stimulate biological material transplanted in a myocardium of a heart during an ejection phase of a cardiac cycle; and
electrically stimulate a chamber of the heart to induce post-extrasystolic potentiation.

13. The medium of claim 12, the instructions further causing the processor to:
electrically stimulate the biological material in response to a first biological signal; and
electrically stimulate the chamber of the heart in response to a second biological signal.

14. A system comprising:
a first electrode configured to deliver a first electrical stimulation to biological material transplanted in a myocardium of the heart;
a second electrode configured to deliver a second electrical stimulation to a chamber of a heart; and
a processor configured to control delivery of the first stimulation during an ejection phase of a cardiac cycle and further configured to control delivery of the second stimulation to induce a post-extrasystolic potentiation.

15. The system of claim 14, further comprising at least one pulse generator configured to generate at least one of the first and second electrical stimulations.

16. The system of claim 14, wherein the first electrical stimulation comprises a set of stimulating pulses.

17. The system of claim 14, wherein the biological material comprises at least one of skeletal myoblasts, differentiated stem cells, undifferentiated stem cells, fibroblasts, endothelial cells, genetically engineered cells, genes and a chemoattractant.

18. The system of claim 14, further comprising a sensor configured to sense a biological signal, wherein the processor is configured to control delivery of the first stimulation in response to the biological signal.

19. The system of claim 18, wherein the sensor is a first sensor and the biological signal is a first biological signal, the system further comprising a second sensor configured to sense a second biological signal, wherein the processor is configured to control delivery of the second stimulation in response to the second biological signal.

20. The system of claim 19, wherein the processor is configured to control delivery of the second stimulation following a waiting period.

21. The system of claim 18, wherein the sensor comprises at least one of an electrode, a blood pressure sensor, an accelerometer, a sonomicrometer, a flow meter, an impedance sensor and a sound sensor.

22. The system of claim 14, wherein the processor is configured to control delivery of the first stimulation following a waiting period.

23. The system of claim 14, wherein the first electrode is configured to be deployed epicardially and the second electrode is configured to be deployed endocardially.

24. The system of claim 14, further comprising a defibrillation module configured to deliver a defibrillation shock to the heart.

25. A system comprising:
first stimulating means for electrically stimulating biological material transplanted in a myocardium of a heart;
second stimulating means for electrically stimulating a chamber of the heart; and
processing means for controlling the first stimulating means to deliver the electrical stimulation when the heart is in the ejection phase, and for controlling the second stimulating means to induce post-extrasystolic potentiation.

26. The system of claim 25, further wherein the processing means is further configured to determine whether the heart is in the ejection phase.

27. The system of claim 25, further comprising sensing means to sense a biological signal, wherein the processing means is further configured to control at least one of the first and second stimulating means as a function of the biological signal.
